SEQUENCE LISTING (15 sequences)

SEQ ID NO: 1 - protein, 20 aa, Artificial Sequence
  Synthetic
SLGPILPPLPGDDSPTTLCS

SEQ ID NO: 2 - protein, 10 aa, Artificial Sequence
  Synthetic
YGRKKRRQRR

SEQ ID NO: 3 - protein, 31 aa, Artificial Sequence
  Synthetic
YGRKKRRQRRXSLGPILPPLPGDDSPTTLCS

SEQ ID NO: 4 - protein, 33 aa, Artificial Sequence
  Synthetic
YGRKKRRQRRGGGSLGPILPPLPGDDSPTTLCS

SEQ ID NO: 5 - protein, 33 aa, Artificial Sequence
  Synthetic
XXYGRKKRRQRRXSLGPILPPLPGDDSPTTLCS

SEQ ID NO: 6 - protein, 35 aa, Artificial Sequence
  Synthetic
XXYGRKKRRQRRGGGSLGPILPPLPGDDSPTTLCS

SEQ ID NO: 7 - protein, 20 aa, Artificial Sequence
  Synthetic
MEEGASGSTPPEELPSPPAS

SEQ ID NO: 8 - protein, 31 aa, Artificial Sequence
  Synthetic
YGRKKRRQRRXMEEGASGSTPPEELPSPPAS

SEQ ID NO: 9 - protein, 33 aa, Artificial Sequence
  Synthetic
YGRKKRRQRRGGGMEEGASGSTPPEELPSPPAS

SEQ ID NO: 10 - protein, 33 aa, Artificial Sequence
  Synthetic
XXYGRKKRRQRRXMEEGASGSTPPEELPSPPAS

SEQ ID NO: 11 - protein, 35 aa, Artificial Sequence
  Synthetic
XXYGRKKRRQRRGGGMEEGASGSTPPEELPSPPAS

SEQ ID NO: 12 - protein, 5 aa, Artificial Sequence
  Synthetic
XPXXP

SEQ ID NO: 13 - protein, 5 aa, Artificial Sequence
  Synthetic
LPPLP

SEQ ID NO: 14 - protein, 5 aa, Artificial Sequence
  Synthetic
LPSPP

SEQ ID NO: 15 - protein, 110 aa, Artificial Sequence
  CH2 domain of p66shc
MNLLPPKPKYNPLRNESLSSMEEGASGSTPPEELPSPPASSLGPILPPLPGDDSPTTLCSFFPRMSNLRLANPAGGRPGSKGEPGRAADDGEGIVGAAMPDSGPLPLLQD

(19) United States
(12) Patent Application Publication
(10) Pub. No.: US 2008/0248574 A1
(43) Pub. Date: Oct. 9, 2008

(54) ANTAGONISTS OF THE INTERACTION OF MELANOMA INHIBITORY ACTIVITY (MIA) AND P66SHC

(75) Inventor: Kaikobad Irani, Pittsburgh, PA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: University of Pittsburgh - Of the Commonwealth System of Higher Education, Pittsburgh, PA (US)

(21) Appl. No.: 12/039,440

(22) Filed: Feb. 28, 2008

Related U.S. Application Data
(60) Provisional application No. 60/892,645, filed on Mar. 2, 2007.

(51) Int. Cl. C12N 15/01; C07K 14/00; C12N 15/11; C12N 15/00; C12N 5/06; C12N 15/87

(52) U.S. Cl. 435/446; 530/350; 536/23.1; 435/320.1; 435/363; 435/375; 435/455

(57) ABSTRACT

The invention provides polypeptides, nucleic acids encoding the polypeptides, optionally in a vector, and their use in methods to disrupt the interaction between p66shc and Melanoma Inhibitory Activity (MIA) in a cell and to sensitize a cell to oxidative stress.

### CROSS-REFERENCE TO RELATED APPLICATIONS

This patent application claims the benefit of U.S. Provisional Patent Application No. 60/892,645, filed Mar. 2, 2007, which is incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 6,690 Byte ASCII (Text) file named “702576.ST25.TXT,” created on Feb. 28, 2008.

### BACKGROUND OF THE INVENTION

Malignant melanoma has high metastatic potential, responds poorly to radiation and chemotherapy, and metastatic melanoma cells are less susceptible to apoptosis in vivo than non-metastatic cells (see, e.g., Kim et al., Cancer Lett., 213: 203-212 (2004)).

There exists a desire to provide methods of sensitizing melanoma cells to oxidative stress-related cell death.

### BRIEF SUMMARY OF THE INVENTION

The invention provides a polypeptide comprising (i) SEQ ID NO: 1 or SEQ ID NO: 7 and (ii) a domain that facilitates entry into a cell.

The invention also provides a method of disrupting interaction between p66shc and Melanoma Inhibitory Activity (MIA) in a cell comprising administering the above-described polypeptide, or a nucleic acid encoding the polypeptide, optionally in a vector, to the cell.

Additionally, the invention provides a method of sensitizing a cell to oxidative stress comprising administering the above-described polypeptide, or a nucleic acid encoding the polypeptide, optionally in a vector, to the cell.

### BRIEF DESCRIPTION OF THE DRAWING

FIG. 1 is a graph showing the percentage of cell viability measured by MTT absorbance in HMB2 melanoma cells that were treated with a polypeptide comprising SEQ ID NO: 1 and SEQ ID NO: 2 (TAT-peptide) and H₂O₂.

### DETAILED DESCRIPTION OF THE INVENTION

The expression of MIA correlates with the progression of melanocytic tumors and predicts the response of melanoma to traditional therapeutic therapies. MIA has been shown to promote melanoma metastasis and invasion in vivo. MIA is primarily expressed in malignant melanoma cells, though other cell types, such as other cancer cells (e.g., glioma, gastric cancer, and pancreatic cancer cells), express varying amounts of MIA. In humans, serum MIA is useful in detecting progression of localized melanoma to metastatic disease and for monitoring therapy of advanced melanomas. MIA belongs to a group of small proteins that adopt a single SH3 domain-like structure and was identified as the first secreted protein with a SH3-like domain.

p66shc was identified as a binding partner of MIA. In particular, the collagen homology-2 (CH2) domain, a 110 amino acid stretch at the N-terminus of p66shc, binds the SH3-like domain of MIA. p66shc belongs to the shcA family of adapter proteins and plays a crucial part in governing mammalian lifespan. p66shc-null (p66shc−/−) mice live 30% longer than their wild-type littermates and are resistant to oxidant stimuli. In response to oxidant stimuli, p66shc is phosphorylated on serine 36, which is essential to its function as a governor of cellular reactive oxygen species levels.

The invention is directed to a polypeptide, nucleic acid encoding the polypeptide, vector comprising the nucleic acid, or compositions comprising the same. The invention also is directed to a method of using any of the above to disrupt the interaction between p66shc and MIA in a cell, and a method of using any of the above to sensitize a cell to oxidative stress.

The polypeptide of the invention comprises a portion of the CH2 domain of p66shc that interacts with MIA and disrupts the interaction of p66shc and MIA in a cell. The polypeptide can comprise any portion of the CH2 domain of p66shc suitable to antagonize the interaction of p66shc and MIA. Preferably, the polypeptide comprises at least 10 (e.g., at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or at least 105) contiguous amino acids of the amino acid sequence of the CH2 domain of p66shc. The polypeptide also can comprise the full-length CH2 domain of p66shc (SEQ ID NO: 15).

The polypeptide of the invention can comprise one or more (e.g., 2, 3, 4, 5, or more) mutations, including deletions, additions, and/or substitutions. While not wishing to be bound by any particular theory, it is believed that the two prolines within the XPXXP (SEQ ID NO: 12) motif are critical for binding MIA. Accordingly, polypeptides comprising portions of the CH2 domain that contain the XPXXP (SEQ ID NO: 12) motif are preferred. For example, residues 41-60 of the CH2 domain (SEQ ID NO: 1) contain LPPLP (SEQ ID NO: 13) and residues 20-40 of the CH2 domain (SEQ ID NO: 7) contain LPSPP (SEQ ID NO: 14), which fit the consensus motif. Particularly preferred polypeptides comprise residues 41-60 of the CH2 domain (SEQ ID NO: 1) and/or residues 20-40 of the CH2 domain (SEQ ID NO: 7).

The polypeptide also can comprise a domain that facilitates entry into a cell. Any domain that facilitates entry into a cell can be included, such as a basic residue-rich (BRR) domain. BRR domains are known in the art and include, for example, the amino acid sequence of HIV TAT (SEQ ID NO: 2). TAT peptides have been shown to efficiently enter a wide variety of cells and tissues when administered, for example, systemically.

The polypeptide also can comprise a spacer. For example, the polypeptide can comprise a spacer between the amino acid sequences of the BRR domain and the CH2 domain. Suitable spacers are known in the art and include, for example, a poly-G spacer (e.g., Gly-Gly-Gly). Examples of polypeptides comprising a BRR domain, CH2 domain, and spacer are set forth in SEQ ID NOs: 3, 4, 8, and 9.

The polypeptide of invention can include additional groups, such as fluorescent tags and linkers. For example, to aid in the detection of the polypeptide in a cell, a fluorescent tag can be added to the polypeptide. Any suitable fluorescent tag can be added to the polypeptide, such as fluorescein, fluorescein derivatives (e.g., FITC), and others known in the art. The fluorescent tag can be attached to the polypeptide by way of a linker. Any suitable linker can be added to the polypeptide, such as the 6-aminohexanoic acid (Ahx) linker. Particular embodiments of the polypeptide of the invention including fluorescent tags and linkers are set forth in SEQ ID NOs: 5, 6, 10, and 11.

The invention also provides a nucleic acid (e.g., DNA or RNA) encoding the above-described polypeptide. The nucleic acid can be isolated and purified or synthesized. The nucleic acid can be formulated as naked DNA or RNA, in a liposome, in recombinant, heat inactivated yeast, or in a vector, such as a plasmid or viral vector. Suitable viral vectors include, but are not limited to, retroviral, adenoviral, lentiviral, adeno-associated, pox, and herpes viral vectors.

The invention also provides an isolated cell comprising the nucleic acid, optionally in a vector, as well as a method of using such a cell to produce the polypeptide.

The inventive polypeptide, nucleic acid, or vector can be used to disrupt interaction between p66shc and MIA in a cell. The method comprises administering an effective amount of the polypeptide, nucleic acid, or vector to the cell. By “effective amount” is meant an amount of the polypeptide, nucleic acid, or vector that is effective to disrupt interaction between p66shc and MIA in a cell. The disruption of the p66shc/MIA interaction allows the available p66shc to interact with other molecules to sensitize the cell to oxidative stress.

Accordingly, the invention also provides a method of sensitizing a cell to oxidative stress comprising administering the polypeptide or nucleic acid encoding the polypeptide, optionally in a vector, to the cell. The cell can be any suitable cell, such as a gastric cancer cell, a pancreatic cancer cell, a melanoma cell, or a glioma cell. Each of these cells expresses varying amounts of MIA. As shown in Example 4, the administration of a polypeptide of the invention to malignant melanoma cells (which express MIA) results in the sensitizing of the cells to oxidative stress.

The polypeptide, nucleic acid, or vector can be administered alone or in a composition (e.g., pharmaceutical composition). When administered in a composition, the composition can comprise any suitable carrier (e.g., pharmaceutically acceptable carrier). The term “pharmaceutically acceptable” refers to a non-toxic material that does not interfere with the effectiveness of the biological activity of the active ingredients. The characteristics of the carrier to be added to the composition depend on the particular route of administration of the pharmaceutical composition to be employed. Such a composition additionally can contain diluents, fillers, salts, buffers, stabilizers, preservatives, antioxidants, solubilizers, and other materials known in the art.

Various routes of administering the polypeptide, nucleic acid, vector, or composition are available. If the polypeptide, nucleic acid, vector, or composition is administered to a mammal (e.g., mouse, rat, hamster, guinea pig, rabbit, cat, dog, pig, cow, horse, or primate, including humans), the polypeptide, nucleic acid, vector, or composition can be administered by, for example, oral, aerosol, parenteral, subcutaneous, intravenous, intramuscular, intraarterial, intrathecal, interperitoneal, rectal, and vaginal administration. The particular pharmaceutical carrier employed will depend, in part, upon the particular polypeptide, nucleic acid, vector, or composition, and the chosen route of administration.

When the polypeptide, nucleic acid, vector, or composition is administered orally, the polypeptide, nucleic acid, vector, or composition can be in the form of a tablet, capsule, powder, solution, or elixir. When administered in the tablet form, the pharmaceutical composition can additionally contain a solid carrier, such as gelatin or an adjuvant.

When the polypeptide, nucleic acid, vector, or composition is administered in liquid form, a liquid carrier such as water, petroleum, oils of animal or plant origin (e.g., peanut oil, mineral oil, soybean oil, sesame oil, or synthetic oils) can be added. The liquid form of the pharmaceutical composition can also contain physiological saline solution, dextrose, or other saccharide solution, and/or glycols, such as ethylene glycol, propylene glycol, or polyethylene glycol.

When the polypeptide, nucleic acid, vector, or composition is administered by parenteral injection (e.g., intravenous, cutaneous, or subcutaneous injection), the polypeptide, nucleic acid, vector, or composition is in the form of a pyrogen-free, parenterally acceptable aqueous solution. The preparation of such parenterally acceptable solutions, with the proper pH, isotonicity, stability, and the like, is within the skill in the art. A preferred pharmaceutical composition for parenteral injection can contain, in addition to the polypeptide, an isotonic vehicle such as Sodium Chloride Injection, Ringer's Injection, Dextrose Injection, Dextrose and Sodium Chloride Injection, Lactated Ringer's Injection, or other vehicles known in the art.

The methods of the invention can further comprise the administration of a chemotherapeutic agent to the cell. Suitable chemotherapeutic agents are known in the art and depend on the particular cancer cell which is targeted. Examples of suitable chemotherapeutic agents include, but are not limited to, cisplatin, 5-fluorouracil (5-FU), doxetaxel, oxaliplatin, epirubicin, leucovorin, mitomycin C, methotrexate, etoposide, paclitaxel, irinotecan, capecitabine, gemcitabine, erlotinib, decarbazine (DITC), vinblastine, temozolomide, and combinations thereof.

Additionally or alternatively, the method can further comprise exposing the cell to radiation. Radiation therapy (radiotherapy) uses high doses of radiation to kill cancer cells and is well-known in the art.

The following examples further illustrate the invention but, of course, should not be construed as in any way limiting its scope.

### EXAMPLE 1

This example demonstrates the ability of p66shc to interact with MIA in vitro.

To identify novel binding partners of p66shc, recombinant CH2 domain of p66shc was used as bait in an in vitro screening assay. In particular, an array (TransSignal SH3 Domain Array III) spotted with the SH3 domains of several proteins (Panomics, Redwood City, Calif.) was used. Recombinant (His)B_{6B}-tagged CH2 domain of p66shc was induced and purified from E. coli, as described in Kasuno et al., Cell Death and Differentiation, 14: 1414-1421 (2007), and incubated with each of the SH3 domains per the array's manufacturer's instructions. Using far-Western blotting of the array, the SH3-like domain of MIA was identified as a strong binding partner of the CH2 domain.

This experiment demonstrates the ability of p66shc to interact with MIA in vitro.

### EXAMPLE 2

This example demonstrates the ability of p66shc to interact with MIA in vivo.

COS-7 cells were co-transfected with cloning vectors comprising either MIA or p66shc and a fluorescent protein. MIA was cloned into the DsRed vector (Clontech, Palo Alto, Calif.) and p66shc was cloned into the EGFP vector (Clonetech, Palo Alto, Calif.). Red and green fluorescence images were captured on a Carl Zeiss Axiovert 200 Fluorescence microscope and merged. Nuclei were counterstained with Hoechst 33342. Ectopic MIA and p66shc expressed in COS7 cells colocalized primarily in the peri-nuclear region.

To determine whether endogenous MIA and p66shc associate with each other in vivo, the human metastatic melanoma cell line HMB2 that expresses MIA, and its derivative cell lines, HMB2-5 in which endogenous MIA expression is suppressed with stable expression of a MIA anti-sense construct, and HMB2-LacZ that stably expresses a control LacZ construct were used. Fractionation of homogenates from MIA-expressing HMB2 cells demonstrated that MIA was principally present in the endoplasmic reticulum, which also had the majority of p66shc.

Immunoprecipitation of endogenous MIA in HMB2 and HMB2-LacZ cells co-precipitated p66shc. The lack of MIA in HMB2-5 cells led to no precipitation of p66shc. Similarly, in HEK 293 cells that do not express MIA, MIA antibody did not pull down endogenous p66shc. In contrast, MIA co-precipitated with p66shc and the isolated CH2 domain of p66shc when expressed in COS7 cells.

To assess whether particular proline residues in the CH2 domain of p66shc mediate its binding to MIA, the binding of MIA with wild-type p66shc and p66shc mutated at proline residues 47 and 50 (p66shcP47A/P50A) of the CH2 domain was compared. Compared to wild-type p66shc, the binding of p66shcP47A/P50A was significantly diminished, indicating that these particular proline residues could be involved in binding.

These experiments demonstrate the ability of p66shc to interact with MIA in vivo.

### EXAMPLE 3

This example demonstrates the ability of MIA to modulate p66shc-mediated H₂O₂ generation and cell death.

In COS7 cells, overexpression of p66shc enhanced H₂O₂-stimulated cell death, which was rescued by co-expression of MIA. Similarly, expression of p66shc in p66shc−/− mouse embryo fibroblasts (MEF) promoted H₂O₂-stimulated death that was rescued by co-expression of MIA. In contrast to wild-type p66shc, expression of the non-phosphorylatable p66shcS36A mutant in p66shc−/− MEF did not increase H₂O₂-stimulated cell death. MIA did not suppress H₂O₂-induced death in p66shc−/− cells expressing p66shcS36A.

In parallel with these findings, expression of p66shc in p66shc−/− MEF led to an increase in endogenous H₂O₂, and co-expression of MIA inhibited this increase. In contrast, in p66shc−/− MEF in which exogenous p66shc was not expressed, MIA did not have any effect on basal endogenous H₂O₂, whereas in p66shc+/+ MEF (wild-type MEF), MIA expression markedly reduced endogenous H₂O₂ levels.

These findings demonstrate that MIA suppresses H₂O₂ levels only in cells that express p66shc, and the capacity of MIA to suppress p66shc-stimulated H₂O₂ and inhibit oxidative stress-induced cell death is only apparent in the context of phosphorylatable p66shc. MIA has no effect on H₂O₂ levels and cell viability in the context of phosphorylation-deficient, functionally inactive, p66shc.

To determine the role of endogenous MIA in regulating cell survival and reactive oxygen species levels, H₂O₂ levels and susceptibility to oxidative stress-mediated death were compared in HMB2 (expresses MIA), HMB2-5 (endogenous MIA expression is suppressed with stable expression of a MIA anti-sense construct), and HMB2-LacZ (stably expresses a control LacZ construct) cells. Basal, steady-state endogenous H₂O₂ levels were significantly higher in the MIA-deficient HMB2-5 cells when compared to HMB2 and HMB2-LacZ. In addition, H₂O₂-induced cell death was markedly greater in HMB2-5 cells when compared to the other two cell lines. Forced reconstitution of MIA in MIA-deficient HMB2-5 cells led to a partial rescue of H₂O₂-induced cell death. Ultraviolet radiation (UVR), which is another stimulus that leads to cell death through oxidative stress, also resulted in a much higher proportion of death in HMB2-5 cells, as compared to the MIA-expressing HMB2 and HMB2-LacZ cells.

Thus, the expression of MIA modulates p66shc-mediated H₂O₂ generation and cell death.

### EXAMPLE 4

This example demonstrates the ability of the polypeptide of the invention to antagonize the function of MIA in malignant cancer cells.

The ability of a polypeptide comprising SEQ ID NO: 4 to enter and sensitize MIA-expressing HMB2 human metastatic melanoma cells to oxidative stress-induced death was examined. Cells were treated with 10 μM FITC-labeled polypeptide, followed by phase contrast and image capture. 2.5 hours after addition of the polypeptide, the majority of the cells were transduced with the peptide.

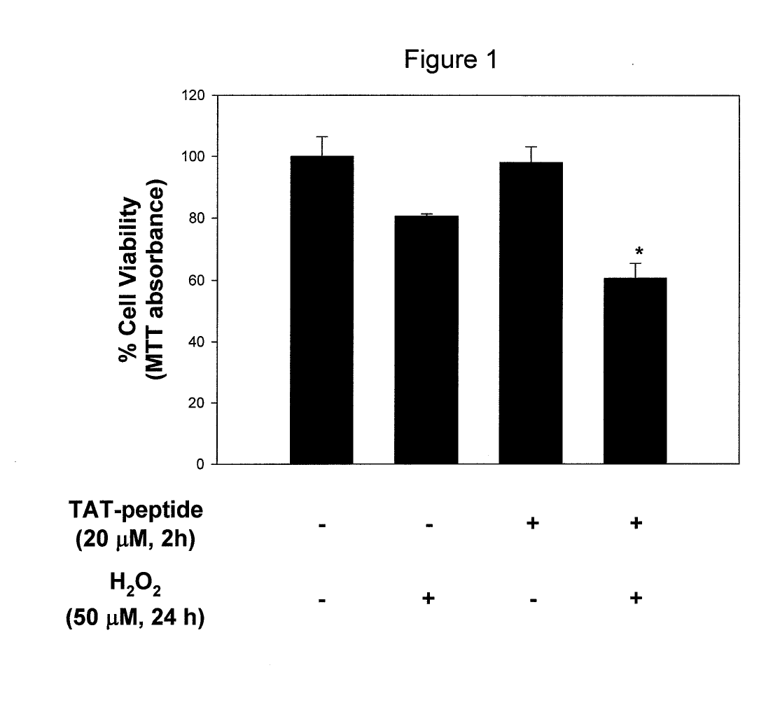

To determine the effect of the administration of a polypeptide comprising SEQ ID NO: 1 on H₂O₂-induced cell death, HMB2 cells were administered 20 μM of the polypeptide. Two hours following the polypeptide administration, the cells were treated with 50 μM of H₂O₂. After 24 hours, the cell viability was quantified using MTT absorbance. As shown in FIG. 1, there was a significant increase in sensitivity of the normally resilient metastatic melanoma cells to H₂O₂-death following administration of the polypeptide of the invention. In particular, when compared to H₂O₂-treated cells in the absence of polypeptide, the sensitivity of H₂O₂-treated cells in the presence of polypeptide was significantly increased (p<0.05).

These experiments demonstrate the ability of the polypeptides of the invention to sensitize the malignant cancer cells to oxidative stress-induced cell death.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A polypeptide comprising (i) SEQ ID NO: 1 or SEQ ID NO: 7 and (ii) a domain that facilitates entry into a cell.

2. The polypeptide of claim 1, wherein the domain that facilitates entry into a cell is a basic residue-rich sequence.

3. The polypeptide of claim 2, wherein the domain comprises SEQ ID NO: 2.

4. The polypeptide of claim 1 further comprising a fluorescent tag, a linker, a spacer, or a combination thereof.

5. The polypeptide of claim 1, wherein the polypeptide comprises an amino acid sequence selected from the group consisting of SEQ ID NO: 3, SEQ ID NO: 4, SEQ ID NO: 8, and SEQ ID NO: 9.

6. A nucleic acid encoding the polypeptide of claim 1.

7. A vector comprising the nucleic acid of claim 6.

8. An isolated cell comprising the nucleic acid of claim 6, optionally in a vector.

9. A method of disrupting interaction between p66shc and Melanoma Inhibitory Activity (MIA) in a cell comprising administering a polypeptide comprising (i) SEQ ID NO: 1 or SEQ ID NO: 7 and (ii) a domain that facilitates entry into a cell, or a nucleic acid encoding the polypeptide, optionally in a vector, to the cell.

10. The method of claim 9, wherein the cell is selected from the group consisting of a gastric cancer cell, a pancreatic cancer cell, a melanoma cell, or a glioma cell.

11. A method of sensitizing a cell to oxidative stress comprising administering a polypeptide comprising (i) SEQ ID NO: 1 or SEQ ID NO: 7 and (ii) a domain that facilitates entry into a cell, or a nucleic acid encoding the polypeptide, optionally in a vector, to the cell.

12. The method of claim 11, wherein the cell is selected from the group consisting of a gastric cancer cell, a pancreatic cancer cell, a melanoma cell, or a glioma cell.

13. The method of claim 11, further comprising administering a chemotherapeutic agent to the cell.

14. The method of claim 11, further comprising exposing the cell to radiation.

15. A method of sensitizing a cell to oxidative stress comprising disrupting the interaction between p66shc and Melanoma Inhibitory Activity (MIA) within the cell.
